(19) United States
(12) Patent Application Publication
(10) Pub. No.: US 2022/0387338 A1
(43) Pub. Date: Dec. 8, 2022

(54) Hydrogel-like particles, methods and uses thereof

(71) Applicant: Association for the Advancement of Tissue Engineering and Cell Based Technologies & Therapies A4tec, Braga (PT)

(72) Inventors: Daniel BARREIRA RODRIGUES, Vila Fonche (PT); Helena ROCHA MOREIRA, Porto (PT); Luca GASPERINI, Braga (PT); Mariana TEIXEIRA CERQUEIRA, Vila Nova de Gaia (PT); Alexandra Margarida PINTO MARQUES, Porto (PT); Rogério Pedro PIRRACO, Porto (PT); Rui Luís GONÇALVES DOS REIS, Porto (PT)

(21) Appl. No.: 17/765,181

(22) PCT Filed: Oct. 2, 2020
(86) PCT No.: PCT/IB2020/059275

§ 371 Date: Mar. 30, 2022

(30) Foreign Application Priority Data
Oct. 4, 2019 (PT) 115817
Dec. 31, 2019 (EP) 19220285.1

(51) Int. Cl. A61K 9/51; C07K 16/28; A61K 38/20

(52) U.S. Cl. CPC A61K 9/5161; A61K 9/5169; C07K 16/2809; C07K 16/2818; A61K 9/5192; A61K 38/2013; A61K 38/208; A61K 38/2086; A61K 38/20

(57) ABSTRACT

The present disclosure relates to a composition for the use in the fields of cancer, immunotherapy and biotechnology. Particularly it relates to the field of gellan gum hydrogel-like particles for artificial antigen presentation in immunotherapy.

### TECHNICAL FIELD

The present disclosure relates to a composition for the use in the fields of cancer, immunotherapy and biotechnology. Particularly it relates to the field of gellan gum hydrogel-like particles for artificial antigen presentation in immunotherapy.

### BACKGROUND

Gellan Gum (GG) is a Sphingomonas elodea exopolysaccharide exhibiting remarkable properties like its biocompatible nature, low production costs and reproducibility over batches. It presents a high resemblance with the extracellular matrix glycosaminoglycan composition which has made it effective in various pharmaceutical and biomedical fields such as in the production of oral formulations (Cox et al. 1999; Shiyani et al. 2009; Agnihotri et al. 2006; Rajinikanth et al. 2007), gellan microbeads (Ahuja et al. 2010; Narkar et al. 2010; Vijan et al. 2012) and ophthalmic formulations (Kesavan et al. 2010; Liu et al. 2010; Singh et al. 2009) for the sustained delivery of relevant drugs. Equally it has also gathered quite some attention in the field of tissue engineering and regenerative medicine (Cerqueira et al. 2014; da Silva et al. 2014; da Silva et al. 2017; Oliveira et al. 2010).

U.S. Pat. No. 8,389,012B2 discloses gellan-gum nanoparticles and methods of making and using the same. Patent US20160325017A1 discloses gellan gum spongy-like hydrogel, its preparation and biomedical applications thereof.

These facts are disclosed in order to illustrate the technical problem addressed by the present disclosure.

### GENERAL DESCRIPTION

Cancer is a global burden, which despite the several clinical and research efforts, still leads to several million deaths per year worldwide. To address this issue, immunotherapy has been presented as an alternative treatment for promoting delayed tumor growth and therefore tumor regression.

When looking into a host immune response towards a solid tumor, dendritic cells (DCs) represent a unique class of antigen-presenting cells (APCs) capable of sensitizing T-cells to both novel or previously contacted antigens. This knowledge has been used in order to develop DC-based cancer immunotherapy strategies, which consists in the generation of cytotoxic effector immune cells. Despite these efforts, current approaches still stand far from ideal with several patients that fail to respond to these types of treatment. One of the main explanations to these issues is the ease with which cells within the tumoral microenvironment have to be immuno-suppressive hindering effective anti-tumoral responses.

The concept of artificial antigen presentation is a viable method to overcome many of the issues regarding immunosuppression and immune-editing, since their function is not modulated by tumor microenvironment.

The present disclose relates to the use of GG for the production of aAPCs, considering the aforementioned resemblance with the extracellular matrix glycosaminoglycan composition which makes it a more “natural origin” material. This main characteristic proves to be advantageous when compared to other aAPCs in the market which comprise liposomes, PLGA based particles, paramagnetic iron-dextran nanoparticles, polystyrene particles, among others. The fact that GG solutions are known to easily crosslink in divalent cationic solutions and form hydrogels, which can withhold high contents of water, is also an advantage of this material.

Avidin is a tetrameric biotin-binding protein produced in the oviducts of birds, reptiles and amphibians and deposited in the whites of their eggs. Dimeric members of the avidin family are also found in some bacteria.[1] In chicken egg white, avidin makes up approximately 0.05% of total protein (approximately 180 μg per egg). The tetrameric protein contains four identical subunits (homotetramer), each of which can bind to biotin (Vitamin B7, vitamin H) with a high degree of affinity and specificity. The dissociation constant of the avidin-biotin complex is measured to be KD≈10-15 M, making it one of the strongest known non-covalent bonds.

Neutravidin protein is a deglycosylated version of avidin, with a mass of approximately 60,000 daltons. As a result of carbohydrate removal, lectin binding is reduced to undetectable levels, yet biotin binding affinity is retained because the carbohydrate is not necessary for this activity. Avidin has a high pI but NeutrAvidin has a near-neutral pI (pH 6.3), minimizing non-specific interactions with the negatively-charged cell surface or with DNA/RNA. Neutravidin still has lysine residues that remain available for derivatization or conjugation.

Streptavidin is a 52.8 (tetramer) kDa protein purified from the bacterium Streptomyces avidinii. Streptavidin homo-tetramers have an extraordinarily high affinity for biotin (also known as vitamin B7 or vitamin H). With a dissociation constant (Kd) on the order of ≈10-14 mol/L,[1] the binding of biotin to streptavidin is one of the strongest non-covalent interactions known in nature. Streptavidin is used extensively in molecular biology and bionanotechnology due to the streptavidin-biotin complex's resistance to organic solvents, denaturants (e.g. guanidinium chloride), detergents (e.g. SDS, Triton), proteolytic enzymes, and extremes of temperature and pH.

In an embodiment the present disclosure comprises a hydrogel-like particle wherein a hydrogel is selected from a list consisting of: gellan gum, hyaluronan, pectin, or mixtures thereof; at least a biotin binding affinity protein bound to the gellan gum particle; at least a biotinylated antibody bound to the biotin binding affinity protein; wherein the biotinylated antibody is able to bind and target an antigen.

In another embodiment, the hydrogel-like particle encapsulates an active ingredient. For the scope of the present disclosure, an active ingredient is defined as a pharmaceutical drug or pesticide that is biologically active.

In an embodiment, the hydrogel-like particle is gellan gum.

In a further embodiment the composition of the present disclosure comprises a biotin binding affinity protein, which is avidin, neutravidin, streptavidin or a combination thereof.

In a further embodiment the composition of the present disclosure comprises a binding affinity protein, which is neutravidin, streptavidin; and a gellan gum particle.

In a particular embodiment the composition of the present disclosure comprises:

5-60% w_{gellan gum}/wₜₒₜₐₗ of the gellan gum nanoparticle, preferably 20-40% w_{gellan gum}/wₜₒₜₐₗ;
1-20% w_{antibody}/wₜₒₜₐₗ of the biotinylated antibody, preferably 10-20% w_{antibody}/wₜₒₜₐₗ.

In a particular embodiment of the present disclosure the active ingredient is a cytokine. In a further embodiment, the cytokine is selected from IL-2, IL-12, IL-15, IL-17 or IL-23.

In a particular embodiment the composition of the present disclosure comprises: 5-60% w_{gellan gum}/wₜₒₜₐₗ of a gellan gum nanoparticle, preferably 20-40% w_{gellan gum}/wₜₒₜₐₗ.

In another embodiment, the composition comprises 0.00001-1% wₚᵣₒₜₑᵢₙ/wₜₒₜₐₗ of biotin binding affinity protein, preferably 0.05-0.1% wₚᵣₒₜₑᵢₙ/wₜₒₜₐₗ;

In another embodiment, the composition comprises 1-20% w_{antibody}/wₜₒₜₐₗ of antibody, preferably 10-20% w_{antibody}/wₜₒₜₐₗ.

In yet another embodiment the composition comprises 10-50% w_{active ingredient}/wₜₒₜₐₗ of active ingredient, preferably 10-40% w_{active ingredient}/wₜₒₜₐₗ;

In an embodiment the composition of the present disclosure comprises:

5-60% w_{gellan gum}/wₜₒₜₐₗ of a gellan gum nanoparticle, preferably 20-40% w_{gellan gum}/wₜₒₜₐₗ;
10-50% w_{cytokine}/wₜₒₜₐₗ of a cytokine, preferably 10-40% w_{cytokine}/wₜₒₜₐₗ;
1-20% w_{antibody}/wₜₒₜₐₗ of an antibody, preferably 10-20% w_{antibody}/wₜₒₜₐₗ.

In a particular embodiment the composition of the present disclosure comprises a biotinylated antibody, which is α-CD3 and/or α-CD28.

In a particular embodiment the composition of the present disclosure comprises a linker between the nanoparticle and the biotin binding affinity protein, which is 1-ethyl-3-(3-dimethylaminopropyl)-carbodiimide (EDC)/N-hydroxysuccinimide (NHS) or avidin/biotin.

In a further embodiment the composition of the present disclosure comprises a size of gellan gum particle from 100-200 nm, in particular 120-150 nm.

In a further embodiment the composition of the present disclosure comprises suitable amounts of a pharmaceutically acceptable excipient.

In a further embodiment the composition of the present disclosure comprises an injectable formulation, in particular an intravenous injection.

In a further embodiment the composition of the present disclosure is to be used in veterinary or human medicine.

In a further embodiment the composition of the present disclosure is to be used in immunotherapy.

In a further embodiment the composition of the present disclosure is to be used in the treatment of cancer diseases, in particular solid tumours.

In a further embodiment the composition of the present disclosure is to be used in the treatment of breast cancer, lymphomas, brain cancer, kidney cancer, liver cancer, lung cancer, or pancreatic cancer.

In a further embodiment the composition of the present disclosure comprises nanoparticles as particles.

In a further embodiment the composition of the present disclosure is comprised by an artificial antigen presenting platform.

In a further embodiment the disclosure encompasses a method to obtain the composition of the present disclosure comprising the following steps:
mixing gellan gum particles with a buffer, preferably 1 mg of particles with 1 ml of buffer;
activating groups of water-soluble carbodiimide/organic compound;
adding a biotin binding affinity protein to the previous solution, so that it adheres to the surface of the activated gellan gum particles, preferably 500 mg of biotin binding affinity protein for each mg of particles;
adding biotinylated antibodies to previous solution/dispersion;
incubating the solution/dispersion obtained in the previous step at a temperature between around 0° C.-10° C.

In a further embodiment the disclosure encompasses a method to obtain the composition of the present disclosure comprising the following steps:
washing the gellan gum particles to remove the excess and unreacted chemical agents; and washing to remove the excess of/unreacted biotin binding affinity protein.

The present disclosure comprises an embodiment, wherein the method to obtain the composition of the present disclosure comprises a biotin binding affinity protein, which is neutravidin, streptavidin or a combination thereof.

The present disclosure comprises an embodiment, wherein the method to obtain the composition of the present disclosure comprises the buffer 2-(N-morpholino)ethanesulfonic acid.

In a further embodiment, the method of the present disclosure comprises groups of water-soluble carbodiimide/organic compound, which are 1-ethyl-3-(3-dimethylaminopropyl)-carbodiimide (EDC)/N-hydroxysuccinimide (NHS) groups.

In a further embodiment, the method of the present disclosure comprises the biotinylated antibody, which is α-CD3 and/or α-CD28 antibodies.

In a further embodiment, the method of the present disclosure comprises an incubation temperature of 4° C.

In a further embodiment, the method of the present disclosure additionally comprises the step of removing unbound antibodies.

Another aspect of the present disclosure relates to the use of the composition described in the present disclosure for in-vitro amplification of T-cells.

### BRIEF DESCRIPTION OF THE DRAWINGS

The following figures provide preferred embodiments for illustrating the disclosure and should not be seen as limiting the scope of invention.

FIG. 1—Schematic illustration representing the process to generate the gellan gum-based particles through an emulsion procedure. Low-acyl gellan gum powder (1) is dissolved at 90° C. for 20 min (2) to give origin to GG solution (w₁) at a predefined concentration (3). Temperature of the dissolved GG is then decreased and added to Chlorophorm and Span80 (O) (4). The mixture is then added in a dropwise manner to a PVA solution (W₂) (5) giving origin to W₁/O/W₂ (6) and afterwards added dropwise to CaCl₂) (7). At this point the solution is crosslinked giving origin to GG nanosphere hydrogels (8) which are then freeze dried (9) originating dried polymeric GG spongy-like hydrogel particles (10).

FIG. 2—Size distribution analysis of GG particles. A) Particle size analysis using Malvern Zetasizer, Nano ZS Series. B) Scanning transmission electron microscopy (STEM) for GG particles.

FIG. 3—Cytotoxicity in primary human dermal fibroblast (hDFbs) cells. A) Dose-response effect of GG nanoparticles on hDFbs. The cells were exposed for 72 h with different particle doses (2, 20, 100 and 200 μg). MTS values were corrected for DNA determination and normalized to the control subset. Data is reported as means±standard deviation of 3 experiments. B) Morphological analysis of hDFbs cells which were plated at 10×10³ cells/well in a 48-well plate and photographed 72 h after plating (magnification ×10).

FIG. 4—Off-the-shelf stability of GG particles. A) Water uptake profile of dried GG particles over 4 days of immersion in PBS. B) Release profile of BSA from GG nanoparticles C) GG particle stability in different solvents analysed with a Malvern Zetasizer, Nano ZS Series, (I) Average size (II) Polydispersity Index of particles. Data are presented as means±standard error, n=3.

FIG. 5—Schematic illustration regarding the fabrication of GG-based aAPCS modified with either functional grade α-CD3 and α-CD28 or biotinylated α-CD3 and α-CD28. GG particles (1) are resuspended in MES buffer 50 mM pH=6.5 (2). A mixture of EDC/NHS in then added to promote terminal group activation (3). Excess and unreacted EDC/NHS is washed out by repetitive centrifugation (4). Neutravidin is then conjugated to the particle surface (5). The particles are then washed again by repetitive centrifugation to remove unreacted Neutravidin (6). Antibodies are then incubated O.N at 4° C. with either functional grade or biotinylated α-CD3/CD28 antibodies (7). Unbound antibodies are removed by several centrifugation washes (8) and functional artificial antigen presenting cells are achieved (9).

FIG. 6—Surface functionalization of GG particles. A) Neutravidin density on the surface of GG particles after chemical binding through EDC/NHS. BI) Characterization of the amount of either functional grade or biotinylated antibodies bound to the surface of GG particles by densitometric analysis. 611) Surface functionalization of GG particles with aCD3 antibody and biotinylated aCD3 were measured by SDS-PAGE. M, molecular mass standards (in kDa; from top to bottom): 250, 130, 100, 70, 55, 35, 25, 15 and 10. 100 μg of GG particles both control (lane 1) or modified (lanes 2-5 and 9-11) or were prepared and heated at 60° C. for 30 min. GG particles were reacted either 500 μg (lanes 2-3) or 1 mg (lanes 4-5) of Neutravidin®. Each condition was respectively incubated with both 10 μg and 20 μg of biotinylated antibody. Note that lanes 2-5 present the 16 kDa subunit of the Neutravidin® protein. A calibration curve of the biotinylated antibody of 0.5 μg, 1 μg and 2 μg was performed (lanes 6-8). The direct binding of the antibody to the particles was also performed (lanes 9 and 10) and again both 10 μg and 20 μg of the antibody were tested. A calibration curve of the standard antibody was equally performed 0.5 μg, 1 μg and 2 μg (lanes 11-13).

FIG. 7—Murine splenocytes were labelled with 5 μM of CFSE for 20 min at 37° C. and then stimulated with both anti-CD3 and anti-CD28 GG particles in a ratio of 1:1 for a period of 7 days. After this period cells were labelled and gated for anti-CD4 and analyzed on a BD FACSAria™ III. Results are presented as the percentage of cells in the final population that have divided. Data are presented as means±standard error.

FIG. 8—Il-2 production by murine splenocytes when stimulated over 7 days with both anti-CD3 and anti-CD28 GG particles in a ratio of 1:1. Control (CTRL) corresponds to the conditions in which unmodified particles were used to stimulate the cell cultures. Results are presented as the percentage of cells in the final population that have divided. Data are presented as means±standard error.

FIG. 9—GG particles tethered to fluorescein probe and visualized under a fluorescent microscope (magnification ×10).

### DETAILED DESCRIPTION

The present disclosure relates to a method to produce submicron GG hydrogel-like particles through an emulsion protocol at the nanoscale, where the nanoparticles may be loaded with cytokines and coated with recombinant molecules in order to prime and expand tumor-specific T-cell responses. This will allow not only the expansion of ongoing T-cell responses but also to modulate their function by means of preventing the development of their “terminal differentiation” or “exhaustion”.

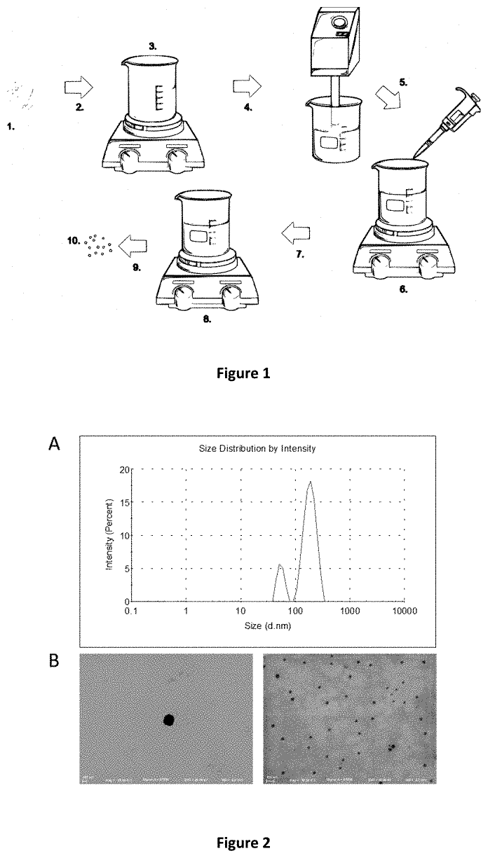

In order to produce such described particles, we used a double emulsion protocol as exemplified in (FIG. 1).

In an embodiment, upon in depth characterization we could confirm the production of particles within the nanometer range in terms of dimension, with and a Z-average (d·nm) of 133.6 (FIG. 2).

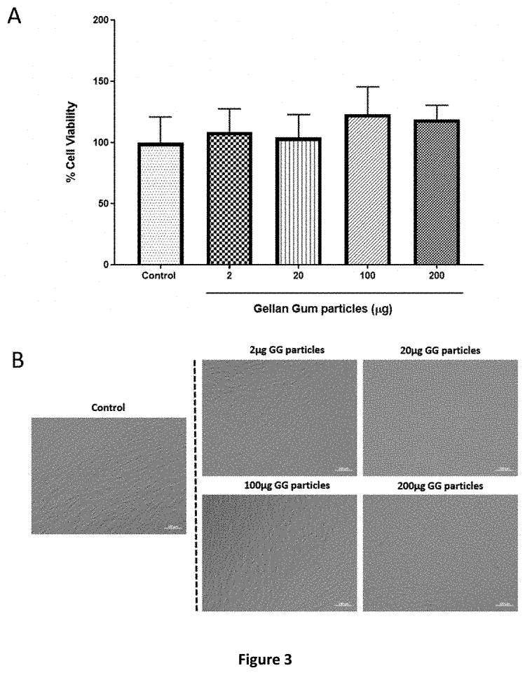

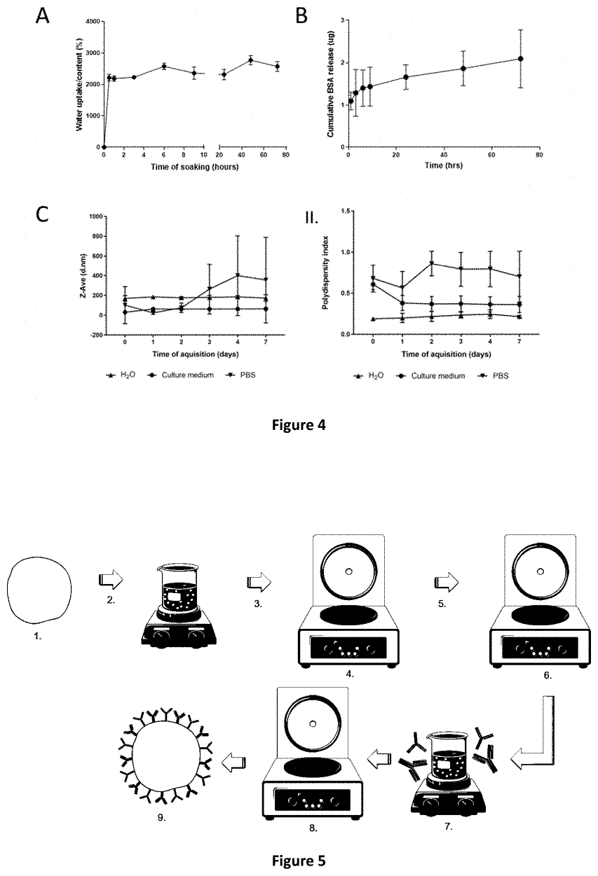

In an embodiment, the biocompatibility and cytotoxicity of the produced GG nanoparticles was also evaluated by means of metabolic assays and cell morphology analysis (FIG. 3). GG particles did not show any effect on regular metabolic behavior or cell morphology.

In an embodiment, the capability of the produced nanoparticles to share similar properties to GG spongy-like hydrogels regarding the high percentage of water uptake after freeze drying was analyzed. To that end, the freeze-dried GG nanoparticles were immersed in H₂O (rehydration step). A rapid weight gain due to the water uptake was observed, with levels between 2700%-3200% (FIG. 4).

Taking this into consideration, the underlying possibility to be able to uptake biologically relevant molecules in an easy fashion and proceed with their controlled release was present. Freeze-dried GG particles were therefore soaked with 10 μg BSA/mg of particles overnight and release profile was assessed over a period of 3 days where we could observe an initial burst release followed by a steady release of up to 2 μg (FIG. 4B).

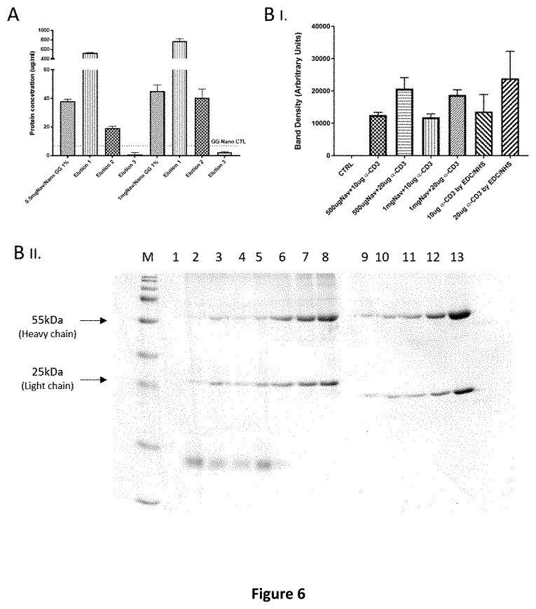

In an embodiment, it was evaluated both the binding potential of neutravidin to the system of the present disclosure as an intermediate for the binding of biotinylated antibodies as well as the grafting of functional antibodies to the system per se (FIG. 6).

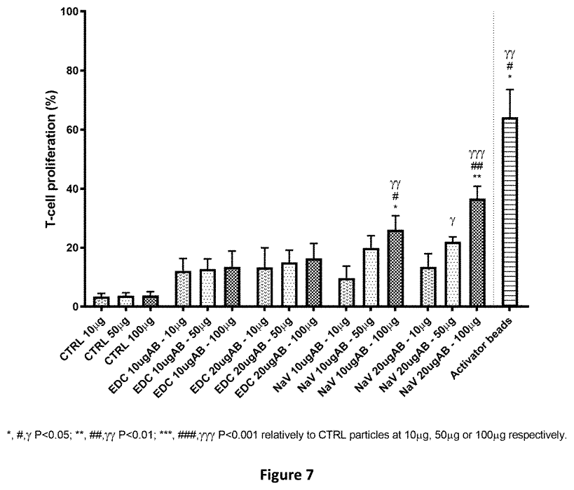

In an embodiment, the functionality of the produced anti-CD3 and anti-CD28 to trigger T-cell proliferation was assessed by in vitro CFSE assays with murine freshly isolated splenocytes. Results are shown in FIG. 7.

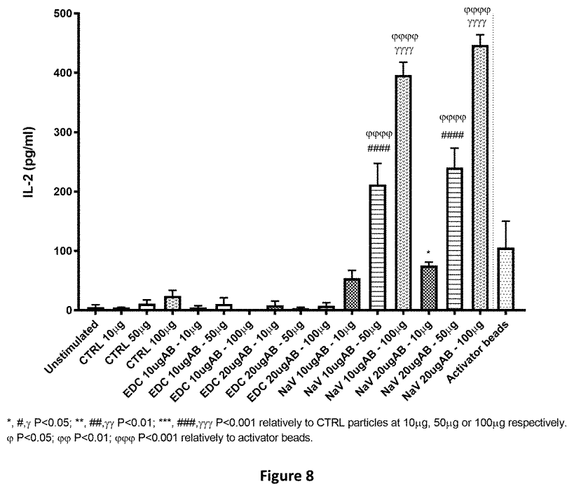

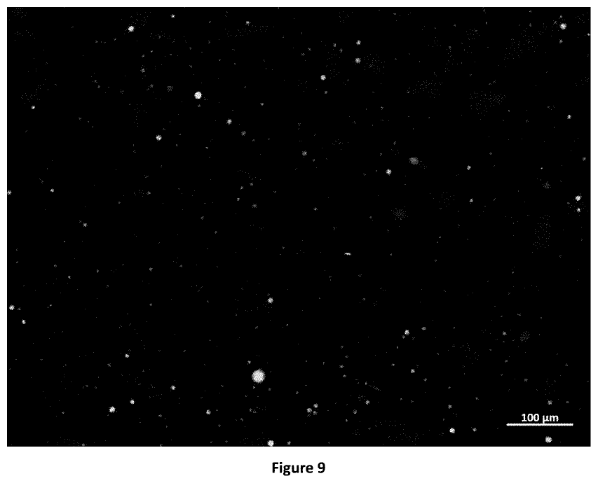

Additionally, to the surface conjugation of biologically relevant molecules or the entrapment by soaking of small molecules for latter release, the aforementioned nanoparticles may be used for tracking by means of conjugating a fluorescent dye to the GG structure previous particle fabrication as can be seen in FIG. 8.

In an embodiment, once the antibody tethering to the GG particles was confirmed, in vitro studies regarding particle and cell interactions were performed. The percentage of CD4+ murine spleen cells which bound to functionalized α-CD3 particles can be shown in the table below (Table 1).

TABLE 1
Interaction of functionalized anti-CD3 GG nanoparticles with murine
CD4⁺ T cells. Results are presented as percentage of cells in the CD4⁺ population that
showed labelling specific for the modified GG nanoparticles. Data are presented as
means ± standard error, n = 2.

| | | Quantity of nanoparticles | % of CD4⁺ cells positive |
|---|---|---|---|
| Days of Stimuli | Experimental conditions | added (μg) | for nanoparticles |
| 1 | GG nanoparticles CTL | 1 | 1.7 ± 0.42 |
| | | 10 | 1.9 ± 0.65 |
| | | 50 | 1.7 ± 0.68 |
| | | 100 | 1.7 ± 0.60 |
| | GG nanoparticles α-CD3 | 1 | 1.6 ± 0.45 |
| | | 10 | 2.23 ± 0.39 |
| | | 50 | 2.6 ± 1.10 |
| | | 100 | 5.8 ± 1.79 |
| | GG nanoparticles NaV α-CD3 | 1 | 2.1 ± 0.52 |
| | | 10 | 4.9 ± 0.96 |
| | | 50 | 10.3 ± 3.81 |
| | | 100 | 18.1 ± 6.25 |
| 5 | GG nanoparticles CTL | 1 | 1.5 ± 1.08 |
| | | 10 | 1.6 ± 1.39 |
| | | 50 | 1.4 ± 0.99 |
| | | 100 | 2 ± 1.72 |
| | GG nanoparticles α-CD3 | 1 | 2.7 ± 2.05 |
| | | 10 | 1.9 ± 1.30 |
| | | 50 | 2.3 ± 1.47 |
| | | 100 | 2.5 ± 1.38 |
| | GG nanoparticles NaV α-CD3 | 1 | 1.6 ± 0.93 |
| | | 10 | 2.8 ± 1.42 |
| | | 50 | 6.4 ± 3.23 |
| | | 100 | 10.6 ± 3.03 |

Upon confirmation that these novel particles would withhold the properties of GG freeze dried hydrogels, it was evaluated their potential as an artificial antigen presenting platform. For this effect the GG nanoparticles were functionalized as presented in the scheme represented in FIG. 5.

The key aspects of the present disclosure are:
Nanoparticles produced from gellan gum through a double emulsion;
Tethering of functional biomolecules (e.g. anti-CD3, anti-CD28, MHCI/MHCII, anti-PD-L1, others);
Encapsulation of small molecules after production by soaking (e.g. IL-2, IL-12, IL-17, IL-23, anti-hypoxia agents, chemotherapeutics, biologics or mixtures thereof);
Conjugation of a fluorescent probe which allows for tracking;
In one system resides an off the shelf approach for artificial antigen presentation, as well as, combination therapy.

In an embodiment for improved results, the measured diameter of the particle can be between 500-8000 Å, preferably 1000-3500 Å.

In an embodiment, the composition may comprise:
5-60% m/m of a GG nanoparticle, preferably 20-40% m/m;
10-50% m/m of a cytokine (IL-2, IL-12, IL-15, IL-17, IL-23), preferably 10-40% m/m;
1-20% m/m of an antibody, preferably 10-20% m/m.

In an embodiment, the fluorescent probe may be selected from DAPI, FITC, RITC, fluorescein-amine, fluorescein, near-infrared dyes or mixtures thereof.

In an embodiment, the linker between the functional group of the polysaccharide and of the biomolecule may be through EDC/NHS or Avidin/Biotin.

In an embodiment, the core nanoparticle should comprise of low-acyl GG, but may also be selected from various negatively charged polysaccharides, such as, alginate, hyaluronan, pectin, or mixture thereof.

In a particular embodiment, the gellan gum particle is a hydrogel-like particle.

The term “comprising” whenever used in this document is intended to indicate the presence of stated features, integers, steps, components, but not to preclude the presence or addition of one or more other features, integers, steps, components or groups thereof.

It will be appreciated by those of ordinary skill in the art that unless otherwise indicated herein, the particular sequence of steps described is illustrative only and can be varied without departing from the disclosure. Thus, unless otherwise stated the steps described are so unordered meaning that, when possible, the steps can be performed in any convenient or desirable order.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

### REFERENCES

1. Agnihotri, S. A., Jawalkar, S. S. & Aminabhavi, T. M., 2006. Controlled release of cephalexin through gellan gum beads: Effect of formulation parameters on entrapment efficiency, size, and drug release. European Journal of Pharmaceutics and Biopharmaceutics, 63(3), pp. 249-261. Available at: http://linkinghub.elsevier.com/retrieve/pii/S0939641106000373 [Accessed Aug. 16, 2017].
2. Ahuja, M., Yadav, M. & Kumar, S., 2010. Application of response surface methodology to formulation of ionotropically gelled gum cordia/gellan beads. Carbohydrate Polymers, 80(1), pp. 161-167. Available at: http://linkinghub.elsevier.com/retrieve/pii/S0144861709006377 [Accessed Aug. 16, 2017].
3. Narkar, M., Sher, P. & Pawar, A., 2010. Stomach-Specific Controlled Release Gellan Beads of Acid-Soluble Drug Prepared by Ionotropic Gelation Method. AAPS PharmSciTech, 11(1), pp. 267-277. Available at: https://doi.org/10.1208/s12249-010-9384-1.
4. da Silva, L. P. et al., 2014. Engineering cell-adhesive gellan gum spongy-like hydrogels for regenerative medicine purposes. Acta Biomaterialia, 10(11), pp. 4787-4797. Available at: http://linkinghub.elsevier.com/retrieve/pii/S1742706114003031 [Accessed Aug. 16, 2017].
5. da Silva, L. P. et al., 2017. Stem Cell-Containing Hyaluronic Acid-Based Spongy Hydrogels for Integrated Diabetic Wound Healing. Journal of Investigative Dermatology, 137(7), pp. 1541-1551. Available at: http://linkinghub.elsevier.com/retrieve/pii/S0022202X17311612 [Accessed Aug. 17, 2017].

1. A composition comprising:
a hydrogel-like particle comprising a substance selected from the group consisting of: gellan gum, hyaluronan, pectin, and mixtures thereof;
a biotin binding affinity protein bound to the hydrogel-like particle;
a biotinylated antibody bound to the biotin binding affinity protein;
wherein the biotinylated antibody is able to bind and target an antigen.

2. The composition of claim 1, wherein the hydrogel-like particle encapsulates an active ingredient.

3. The composition of claim 2, wherein the substance of the hydrogel-like particle is gellan gum.

4. The composition of claim 3, wherein the biotin binding affinity protein is avidin, neutravidin, streptavidin or a combination thereof.

5. (canceled)

6. The composition of claim 1, wherein the hydrogel-like particle is a gellan gum nanoparticle and wherein the composition comprises:
5-60% w_{gellan gum}/wₜₒₜₐₗ of the gellan gum nanoparticle;
1-20% w_{antibody}/wₜₒₜₐₗ of the biotinylated antibody.

7. The composition of claim 2, wherein the active ingredient is a cytokine selected from the group consisting of IL-2, IL-12, IL-15, IL-17, and IL-23.

8. (canceled)

9. (canceled)

10. The composition of claim 1, according to any one of the previous claims comprising: 0.00001-1% wₚᵣₒₜₑᵢₙ/wₜₒₜₐₗ of biotin binding affinity protein.

11. (canceled)

12. The composition of claim 1, comprising: 10-50% w_{active ingredient}/wₜₒₜₐₗ of active ingredient.

13. (canceled)

14. The composition of claim 1, wherein the biotinylated antibody is α-CD3 and/or α-CD28.

15. (canceled)

16. The composition of claim 3, according to any one of the previous claims wherein the size of gellan gum particle is from 100-200 nm.

17. (canceled)

18. (canceled)

19. (canceled)

20. (canceled)

21. The composition of claim 1, wherein the composition is suitable for the treatment of solid tumours.

22. The composition of claim 1, wherein the composition is suitable for the treatment of breast cancer, lymphomas, brain cancer, kidneys cancer, liver cancer, lung cancer, or pancreatic cancer.

23. The composition of claim 1, wherein the particles are nanoparticles.

24. An artificial antigen presenting platform comprising the composition of claim 1.

25. A method to obtain the composition of claim 3, comprising:
mixing gellan gum particles with a buffer;
activating groups of water-soluble carbodiimide/organic compound;
adding a biotin binding affinity protein to the previous solution, so that it adheres to the surface of the activated gellan gum particles;
adding biotinylated antibodies to previous solution/dispersion; and
incubating the solution/dispersion obtained in the previous step at a temperature between around 0° C.-10° C.

26. The method of claim 25, further comprising:
washing the gellan gum particles to remove the excess and unreacted chemical agents; and
washing to remove the excess of/unreacted biotin binding affinity protein.

27. The method of claim 25, wherein the biotin binding affinity protein is neutravidin, streptavidin or a combination thereof.

28. (canceled)

29. The method of claim 25, wherein the groups of water-soluble carbodiimide/organic compound are 1-ethyl-3-(3-dimethylaminopropyl)-carbodiimide (EDC)/N-hydroxysuccinimide (NHS) groups.

30. The method of claim 25, wherein the biotinylated antibody is α-CD3 and/or α-CD28 antibodies.

31. (canceled)

32. The method of claim 25, further comprising the step of removing unbound antibodies.

33. (canceled)
